(19) United States
(12) Patent Application Publication
(10) Pub. No.: US 2016/0265051 A1
(43) Pub. Date: Sep. 15, 2016

(54) Methods for Detection of Fetal Chromosomal Abnormality Using High Throughput Sequencing

(71) Applicant: Yandong Cao, Nanjing (CN)

(72) Inventor: Yandong Cao, Nanjing (CN)

(21) Appl. No.: 14/658,192

(22) Filed: Mar. 15, 2015

(51) Int. Cl. C12Q 1/68; G06F 19/00; G06F 19/22

(52) U.S. Cl. CPC C12Q 1/6883; C12Q 1/6869; G06F 19/22; G06F 19/345

(57) ABSTRACT

Disclosed are methods for non-invasively detecting fetal chromosomal abnormality in maternal samples using high throughput sequencing technologies. The present invention provides a method to minimize the influence of G/C-content in analyzing sequencing data and thus increase the sensitivity and accuracy in detecting any aneuploid chromosome in a genome. This method is especially helpful for analyzing sequencing data with lower quality or low coverage.

### BACKGROUND OF THE INVENTION

1. Field of the Invention

The present invention provides methods to noninvasively detect prenatal chromosomal abnormality. In particular, it provides methods for detecting fetal aneuploidy using high-throughput sequencing of DNA in maternal samples.

2. Description of the Related Art

Aneuploidy is a type of chromosome abnormality that an organism has a deviated copy number from normal for one or more chromosomes. An aneuploid is an individual organism whose chromosome number differs from the wild type by part of a chromosome set. Generally, the aneuploid chromosome set differs from the wild type by only one or a small number of chromosomes. Babies born with aneuploidy will have birth defects such as Down Syndrome, Edward's Syndrome and Patau Syndrome, which is caused by additional copy of chromosome 21, 18 and 13, respectively. Fetal aneuploidy and other chromosomal abnormality affect 9 out of 1000 live births. Conventional diagnosis of fetal aneuploidy is based on karyotyping fetal cells obtained from amniocentesis or chorionic villus sampling. These invasive procedures impose small but potentially significant risk to both mother and fetus, and can potentially result in miscarriages. The discovery of fetal cells and cell-free fetal nucleic acids in maternal blood in the past few decades have prompted researchers to seek for noninvasive methods to diagnose fetal aneuploidy using fetal DNA in maternal blood. However, the small amount of fetal genetic material relative to the maternal counterparts in maternal blood poses a technical challenge for such task.

Recent advances in high-throughput shotgun sequencing enables parallel sequencing of tens of millions of short sequences in maternal plasma DNA sample, of which a small fraction originates from the fetus, followed by mapping these short sequences to the chromosome of origin. These short sequences are called sequence tags as they have sufficient sequence information to allow them to be precisely assigned to the original position on the chromosome of origin. The ability to count millions of DNA sequence tags allowed detection of small changes in the representation of chromosomes contributed by an aneuploid fetus in a maternal plasma sample. The over-representation or under-representation of any chromosome caused by fetal chromosome contribution is detected by comparing the number of sequence tags assigned to a chromosome of interest in a prenatal blood sample to that of normal chromosomes. When the difference between these two numbers is statistically significant, a fetal aneuploid chromosome is detected.

Ideally, sequence tags are equally distributed across each chromosome contributed by genomic DNA from a normal diploid mother and a normal diploid fetus. An aneuploid fetus with more or less copy of one particular chromosome (e.g. chromosome 21) will lead to over- or under-presentation of the particular chromosome in the maternal blood cell-free circulating DNA. The imbalance of the presentation of a particular chromosome can be statistically detected in a high throughput sequencing with tens of millions of sequence tags. However, sequence bias, especially G/C sequence bias, during PCR amplification either at the sample preparation or the sequencing process makes the short sequence reads deviated from ideal counting statistics. Sequences with higher G/C content tend to have higher number of sequence tags fallen in a measuring window of the same length (U.S. Pat. No. 8,195,415). In addition, sequences of different G/C contents have also shown differences in the variance of their sequence reads with higher variation in G/C-rich sequences (e.g. 50-55% G/C). This poses a practical limit on the sensitivity of the test especially for detecting aneuploidy especially associated with G/C-poor or G/C-rich chromosomes. Higher quality control and deeper sequencing coverage is needed to overcome the G/C sequence bias. Fan et al. (PLoS ONE 2010, 5(5): e10439.) describes a method to computationally remove G/C bias in short read sequencing data by applying weight to each sequenced read based on local genomic G/C content. However, sequence reads at G/C-rich regions could still contribute to measurement noise and lower the sensitivity in detecting fetal aneuploidy. The present invention provides a correction method that can minimize G/C bias in analyzing the sequence reads, remove the counting noise attributed to G/C-rich regions, accommodate the analysis of sequencing data with lower quality and increase the sensitivity of detecting fetal aneuploidy in any chromosome of a genome. The method is very robust and can be applied to analyze samples from different sequence runs and from different customers, which enables data collection and data sharing among different end users.

### SUMMARY OF THE INVENTION

High throughput sequencing is used to noninvasively detect fetal aneuploidy by searching for abnormal fetal chromosome distribution in circulating DNA of maternal blood. Sequencing artifacts due to difference of G/C content across the genome is a major reason affecting the sensitivity and accuracy of this method. It is the goal of the present invention to provide a method to minimize the influence of G/C-content in analyzing sequence tags and increase the sensitivity and robustness of this method in detecting aneuploid chromosome especially in sequencing data with lower quality and lower coverage.

The present invention provides a sensitive and robust method for detecting an abnormal distribution of a chromosome or chromosome portion of interest in a mixed DNA sample of normally and abnormally distributed chromosomes by counting sequence tags obtained from a high throughput sequencing of the mixed DNA sample. The method comprises sequencing DNA in the mixed DNA sample with normally and abnormally distributed chromosomes by a high throughput sequencing method to obtain a number of sequence tags of sufficient length to be assigned to a chromosome location within a genome. The mixed DNA sample can be, for example, a maternal DNA sample from blood, urine or saliva, which contains both fetal and maternal DNA. The length of the sequence tags can be, for example, 20 to 200 bp depending on the high throughput sequencing platform used.

Secondly, map the sequence tags to the chromosome of origin by comparing the sequence in the sequence tags to a reference genome. When mapping the sequence tags, one mismatch is allowed to take consideration of polymorphism between the test chromosome and the reference genome. Divide each chromosome into non-overlapping sliding windows of predefined length and determine sequence tag density mapped to each sliding window. The length of the sliding windows can selected from 10 kb to 200 kb, preferably 20 kb to 100 kb. The sliding window is selected such that there are sufficient number of sequence tags falling into each window and there are sufficient number of sliding windows on the chromosome of interest.

Thirdly, calculate G/C-content for each sliding window on the chromosome of interest and group sliding windows into different categories based on G/C-content. Select one or more G/C-content intervals in which majority of sequence tags fall and exclude G/C-content intervals with few sequence tags or low quality data. In a preferred embodiment, the selected G/C-content intervals are 35-40%, 40-45%, 45-50%. For each selected G/C-content interval, determine a mean or median value of the sequence tag density for all the sliding windows within the G/C content interval.

Fourthly, compare the mean or median value of each selected G/C-content interval on the chromosome of interest in the mixed DNA sample to a mean or median value of the same selected G/C-content interval of normally distributed chromosome(s) to obtain a statistic value. An important feature of the present invention is to compare sequence tag densities in the same G/C-content interval so as to minimize the influence of sequencing artifacts caused by the difference of G/C content in the sequence comparisons. To determine the existence of abnormal distribution of the chromosome of interest (first chromosome), the comparison can be made between different chromosome within the same sample or between the same chromosome of different samples. In one embodiment, the mean or median sequence tag density in each selected G/C-content interval of the first chromosome in the testing sample is compared to that of the first chromosome in a different sample or samples with a normal distribution of the first chromosome. To account for difference in sequence tag counts in different samples, the sequence tag density needs to be normalized to the average sequence tag density for all the autosomes in each sample. In another embodiment, the mean or median sequence tag density in each selected G/C-content interval of the first chromosome is compared to the mean or median sequence tag density in the same G/C-content interval of a second chromosome or chromosomes which have a normal distribution in the same test sample. The second chromosomes can be all the autosomes in the testing sample, excluding the first chromosome.

Finally, the statistic value for each G/C-content interval is combined to obtain a weighted statistic value, which is weighted by the percentage of sequence tags in each interval. Determine the existence of abnormal distribution of the chromosome of interest in the test sample based on the weighted statistic value. If the weighted statistic value falls within the boundary of a predefined confidence interval (e.g. 99%), the chromosome of interest in the test sample is considered to be normal. If the weighted statistic value falls outside the boundary of predefined confidence interval (e.g. 99%), the chromosome of interest is considered to be abnormally distributed and an aneuploid chromosome is detected in the test sample.

In another embodiment, the present invention provides a computer program that can detect an abnormal distribution of chromosome or chromosome portion of interest in a mixed DNA sample of normally and abnormally distributed chromosomes by counting sequence tags obtained from a high throughput sequencing of the mixed DNA sample. The computer program takes sequence tag reads of the high throughput sequencing of the mixed DNA sample as the input data. It divides each chromosome of a reference genome into non-overlapping windows and maps the input sequence tags to these windows. It then counts numbers of sequence tags in each window on each chromosome. The number of sequence tags in each window is normalized to the average value of sequence tag counts in all the windows of all the autosomes in the sample. The computer can group the windows based on selected G/C-content intervals for each chromosome and calculate the mean or median value of sequence tag counts in each selected G/C-content interval. The computer makes comparison of the mean or median sequence tag counts between any two chromosomes within the same sample or compare sequence tag counts of chromosome of interest between the test sample and normal samples stored in the system. The computer can also make comparison between one chromosome vs. multiple chromosomes, or between one sample vs. multiple normal samples. The computer outputs a weighted statistic value based on individual statistic values of each selected G/C-content interval and make a decision call on the existence of chromosome abnormality based on a pre-selected confidence interval. The computer program can be used to analyze sequencing data from difference sequencing platforms, for example, Illumina high throughput sequencing platform and Ion Proton high throughput sequencing system from Thermal Fishier Scientific. It supports separate user account and data sharing among different users.

### BRIEF DESCRIPTION OF THE DRAWINGS

FIG. 1 is a scatter plot showing the relationship between any two parameters chosen from G/C content of sequences in a sliding window (GC), Degree of freedom (df), mean of sequence tag reads (mean), median of sequence tag reads (median), weighted mean of sequence tag reads (weight_mean) and weighted median of sequence tag reads (weight_median). The scatter plot shows that sequence G/C content has non-linear relationship with all the other parameters. The influence of G/C content in sequence read analysis cannot be easily compensated by any linear method.

FIG. 2 shows the distribution of human genomic sequence counts within different intervals of G/C content. The whole human genome (hg19) was divided into large fragments/bins of 20 kb, which were grouped into five different categories based on their sequence G/C contents. The G/C contents for the five categories are 30-35%, 35-40%, 40-45%, 45-50% and 50-55%. The x-axis is the count of sequence tags in a bin and the y-axis is the number of bins having the corresponding sequence tag counts. The figure shows that the majority of sequences fall in the three G/C categories: 35-40%, 40-45% and 45-50% and different groups of sequences have different peak values.

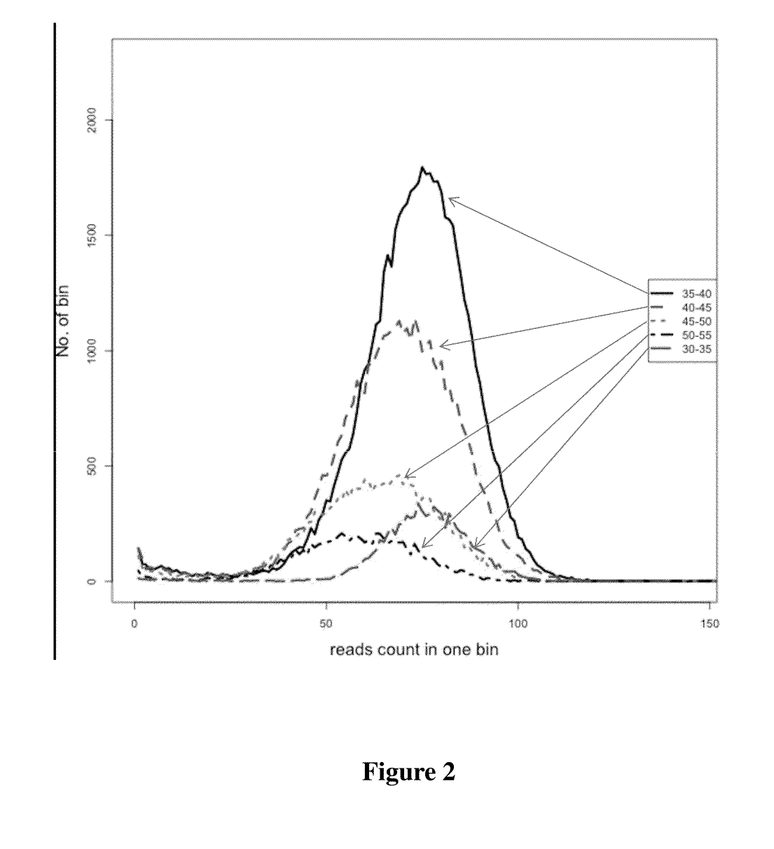

FIG. 3 shows a diagram of an online computer program (iNIPT, Non-Invasive Prenatal Test) workflow for detecting fetal Trisomy 13, 18 and 21 in a maternal sample.

FIG. 4 shows the launching page with parameters setting of computer program iNIPT.

FIG. 5 shows the output and report page of iNIPT. The output page shows the G/C-adjusted Z-score for chromosome 21, 18 and 13 and corresponding probabilities that accept the null hypothesis (the chromosome is normally distributed).

TABLE 1
Detection of Fetal Aneuploidy using Illumina Platform

| | | total_reads | | chr13 | chr18 | chr21 |
|---|---|---|---|---|---|---|
| | adjust | (in millions) | G/C_ratio (%) | (z-score) | (z-score) | (z-score) |
| T13 | | | | | | |
| 1 | GC-adjust | 9.11 | 44.66 | 2.9 | 0.3 | 1.8 |
| 1 | No-GC-adjust | 9.11 | 44.66 | 0.4 | 0 | 0.4 |
| T18 | | | | | | |
| 2 | GC-adjust | 7.05 | 45.98 | −0.7 | 4.8 | 0.9 |
| 2 | No-GC-adjust | 7.05 | 45.98 | −2.1 | 4 | −1 |
| 3 | GC-adjust | 7.86 | 42.53 | −1.7 | 5.9 | 1.1 |
| 3 | No-GC-adjust | 7.86 | 42.53 | −1.8 | 3.7 | 0.2 |
| 4 | GC-adjust | 6.59 | 42.48 | 0.1 | 3.4 | 1.3 |
| 4 | No-GC-adjust | 6.59 | 42.48 | −0.5 | 2.6 | 0.2 |
| 5 | GC-adjust | 8.47 | 45.76 | 0.3 | 3.6 | 2.1 |
| 5 | No-GC-adjust | 8.47 | 45.76 | −1.5 | 2.9 | 0.6 |
| 6 | GC-adjust | 8.2 | 45.42 | 0.1 | 4.3 | 1.9 |
| 6 | No-GC-adjust | 8.2 | 45.42 | −2.1 | 3.1 | 0 |
| 7 | GC-adjust | 14.57 | 40.81 | −1.4 | 4.2 | −0.6 |
| 7 | No-GC-adjust | 14.57 | 40.81 | −0.2 | 4.3 | −1.2 |
| 8 | GC-adjust | 8.13 | 41.2 | 0.9 | 4.6 | 1.5 |
| 8 | No-GC-adjust | 8.13 | 41.2 | 0.6 | 4.7 | 1.7 |
| 9 | GC-adjust | 7.82 | 41.67 | −0.5 | 3.1 | 2.1 |
| 9 | No-GC-adjust | 7.82 | 41.67 | −0.6 | 3 | 1.7 |
| T21 | | | | | | |
| 10 | GC-adjust | 8.47 | 41.66 | −1.3 | −2.1 | 4.3 |
| 10 | No-GC-adjust | 8.47 | 41.66 | −0.6 | −2.3 | 3.7 |
| 11 | GC-adjust | 6.44 | 44.29 | −0.1 | −1.6 | 6.5 |
| 11 | No-GC-adjust | 6.44 | 44.29 | −1.8 | −1.8 | 5.4 |
| 12 | GC-adjust | 6.68 | 44.54 | −0.5 | −0.3 | 7.5 |
| 12 | No-GC-adjust | 6.68 | 44.54 | −1.8 | −0.6 | 5.3 |
| 13 | GC-adjust | 8.68 | 47.61 | 2.9 | 1.4 | 8.9 |
| 13 | No-GC-adjust | 8.68 | 47.61 | −2.5 | 0.4 | 5.5 |
| 14 | GC-adjust | 7.39 | 46.72 | 1.8 | 0.7 | 11.2 |
| 14 | No-GC-adjust | 7.39 | 46.72 | −1.8 | 0 | 7.9 |
| 15 | GC-adjust | 10.87 | 44.36 | −0.3 | −0.6 | 6.2 |
| 15 | No-GC-adjust | 10.87 | 44.36 | −1.5 | −1.1 | 4.2 |
| 16 | GC-adjust | 6.05 | 45.84 | 0.3 | 0.2 | 9.5 |
| 16 | No-GC-adjust | 6.05 | 45.84 | −1.2 | 0.1 | 7.4 |

TABLE 2
Detection of Fetal Aneuploidy using Ion Torren Platform

| | | total_reads | G/C_ratio | chr13 | chr18 | chr21 |
|---|---|---|---|---|---|---|
| | adjustment | (in millions) | (%) | (z-score) | (z-score) | (z-score) |
| T13 | | | | | | |
| 1 | GC-adjust | 8.92 | 42.69 | 3.8 | 1.1 | −0.4 |
| 1 | No-GC-adjust | 8.92 | 42.69 | 1.5 | −0.1 | 1.9 |
| 2 | GC-adjust | 6.82 | 43.2 | 3.6 | 0.7 | 0.2 |
| 2 | No-GC-adjust | 6.82 | 43.2 | 1.7 | 0.4 | 2 |
| 3 | GC-adjust | 6.82 | 44.48 | 5.1 | 1.2 | −1 |
| 3 | No-GC-adjust | 6.82 | 44.48 | 2.1 | 1.5 | 2.1 |
| 4 | GC-adjust | 6.56 | 39.32 | 3.1 | 0.2 | 0 |
| 4 | No-GC-adjust | 6.56 | 39.32 | 1.3 | −0.9 | −0.4 |
| T18 | | | | | | |
| 5 | GC-adjust | 11.28 | 42.4 | −1.8 | 3.5 | −2.1 |
| 5 | No-GC-adjust | 11.28 | 42.4 | 2.3 | 4.1 | 0.5 |
| 6 | GC-adjust | 2.1 | 40.87 | 2 | 5.3 | NA |
| 6 | No-GC-adjust | 2.1 | 40.87 | 2.7 | 4.5 | NA |
| T21 | | | | | | |
| 7 | GC-adjust | 3.67 | 39.39 | 2.5 | 2.5 | 3.1 |
| 7 | No-GC-adjust | 3.67 | 39.39 | −0.2 | 1.5 | 2.1 |
| 8 | GC-adjust | 4.72 | 40.71 | 1 | −0.1 | 3.2 |
| 8 | No-GC-adjust | 4.72 | 40.71 | 0.9 | −0.2 | 1.8 |
| 9 | GC-adjust | 4.46 | 41.22 | 0.5 | 1.1 | 3.1 |
| 9 | No-GC-adjust | 4.46 | 41.22 | 2.2 | −0.4 | 1.2 |
| 10 | GC-adjust | 4.99 | 39.67 | 0.5 | 0.9 | 3.2 |
| 10 | No-GC-adjust | 4.99 | 39.67 | 1.8 | −0.6 | 4 |
| Normal | | | | | | |
| 11 | GC-adjust | 5.77 | 42.6 | −0.2 | 2.3 | 0 |
| 11 | No-GC-adjust | 5.77 | 42.6 | 3.4 | 2.7 | 2.2 |

TABLE 3
Summary of Fetal Aneuploidy Test using Ion Torren Platform

| | Normal | T13 | T18 | T21 |
|---|---|---|---|---|
| Real Value | 59 | 4 | 2 | 4 |
| GC-adjusted | 59 | 4 | 2 | 4 |
| No GC-adjusted | 65 | 0 (1 false positive) | 2 | 1 |

TABLE 4
Summary of Fetal Aneuploidy Test using Illumina Platform

| | Normal | T13 | T18 | T21 |
|---|---|---|---|---|
| Real Value | 30 | 1 | 8 | 7 |
| GC-adjusted | 31 | 0 | 8 | 7 |
| No GC-adjusted | 33 | 0 | 6 | 7 |

### DETAILED DESCRIPTION

### Definitions

Unless otherwise defined, all the technical and scientific terms will be used in accordance with common understanding of persons ordinary skilled in the art to which the present invention is related. For purpose of clarity, the following terms are defined below and shall have the assigned meanings unless a contradictory definition is clearly indicated from the context in which the term is used.

The term “maternal sample”, as used herein, refers to any sample taken from a pregnant mammal which comprises a maternal source and a fetal source of nucleic acids. For example, maternal blood sample, including maternal serum or plasma sample, has DNA from both mother and fetus. Other examples include, but not limited to, maternal urine and saliva samples.

The term “aneuploidy”, as used herein, refers to a condition in which an organism has a chromosome number different from the normal chromosome number of that organism. For example, the organism may have one extra chromosome or a missing chromosome. A normal human has disomic chromosomes with 22 pairs of autosomes and a pair of sex chromosomes. Any person has chromosomes different from normal disomic chromosomes is an aneuploid person. Most common human aneuploidy cases in live births have an extra chromosome at Chr. 21 (Trisomy 21, T21), Chr. 18 (Trisomy 18, T18) or Chr. 13 (Triosomy 13, T13). The definition also includes situations that an organism has an extra or misses a large portion of a chromosome. For example, a partial duplication of the long arm of chromosome 18 (18q.21.1-qter) can result in Edwards Syndrome.

The term “aneuploid chromosome”, as used herein, refers to a chromosome that has an abnormal copy number. It may have one more or less than the normal copy number. For example, chromosome 21, 18 and 13 are common aneuploid chromosomes found in humans. The extra or missing chromosome can be a complete chromosome or a large portion of the chromosome.

The term “high throughput sequencing”, also called massively parallel sequencing or next generation sequencing, refers to non-Sanger-based sequencing technologies, in which millions or tens of millions of short DNA strands can be sequenced in parallel. The output of high throughput sequencing is millions or tens of millions of short sequence reads that can be assigned to specific chromosome locations in a genome. These short sequence strands obtained from high throughput sequencing are usually called “sequence tags”, which are have sufficient length to be uniquely assigned to the chromosome location where they come from.

The term “sequence tags”, as used herein, refers to short sequence reads as outputs of high throughput sequencing that can be assigned to the chromosome location of origin. The sequence tags can be mapped to the chromosome by comparing their sequence with a reference genome. Only sequence tags that can be uniquely assigned to a chromosome are used in the analysis of this invention. The sequence tags usually have a length of 20 to 200 base pairs, and preferably have a length of 20 to 100 base pairs.

The term “sliding window”, as used herein, refers to a window of certain length that slides across contiguous, non-overlapping sequence region to cover the entire length of a chromosome. The average number of sequence tags falling into each sliding window is used as a measure of presentation for a chromosome in a DNA sample. The length of a sliding window is selected such that there are sufficient number of sequence tags (e.g. 20 to 200) in each window and there are sufficient number of windows on each chromosome. The length of a sliding window is usually from 5 kb to 200 kb, and preferably from 20 kb to 100 kb.

The term “sequence tag density”, as used herein, refers to the number of sequence tags that fall in a window of certain length. The sequence tag density of a chromosome is the average number of sequence tags of all the sliding windows on the chromosome. Not considering sequencing or other artifacts, the sequence tag density is theoretically expected to be the same for all normal chromosomes in a sample. The sequence tag density of a chromosome that is significantly different from that of normal chromosomes indicates the over- or under-representation of the particular chromosome.

The term “abnormally distributed chromosome”, as used herein, refers to a chromosome with a copy number distribution that is significantly different from that of a normal chromosome. In particular, it refers to a chromosome with a sequence tag density distribution that is significantly different from that of a normal chromosome in a high throughput sequencing output. For example, in a maternal blood DNA sample with genomic DNA from mother and fetus, contribution from an abnormal fetal chromosome makes the sequence tag density of the abnormal chromosome statistically deviated from that of normal chromosome. A fetal chromosomal abnormality can then be detected by finding abnormally distributed chromosome in the population of DNA with normal and abnormal distribution.

The term “G/C-content interval”, as used herein, refers to a range of G/C-content for a DNA sequence of interest, having an upper and lower limit. It is expressed as a range of the percentage of G/C nucleotides in a DNA sequence. For example, an G/C-content interval for a sequence can be 35% to 40%, 38% to 41%, or 40% to 48%.

### General Description of Principles and Methods

Chromosomal dosage resulting from fetal aneuploidy can be detected using DNA in a maternal blood sample containing DNA from both maternal and fetal sources. However, the small amount of fetal genetic material relative to the maternal counterparts in maternal blood poses a technical challenge for such task. High throughput sequencing generates of millions or tens of millions of short DNA sequence tags in parallel, enabling statistic analysis for detection of the small difference in chromosomal dosage contributed by fetal aneuploid source. This technology uses the sequence tag density of a chromosome as a quantitative measure of chromosomal dosage in a maternal sample. Sequencing artifacts, largely due to difference of sequence G/C-content across a genome, results in sequence counting deviated from ideal statistics, increases variation of sequencing reads and decreases the sensitivity of detection limits. It also requires to have more stringent quality control and deeper coverage in the high throughput sequencing to overcome the influence of G/C bias, which significantly increases the cost of such a test. The present invention provides a method to minimize the influence of G/C-content in analyzing sequence tag counts and increase the sensitivity and robustness of this method in detecting aneuploid chromosome especially with sequencing data of lower quality.

The conventional method for calculating the sequence tag density of a chromosome uses the average number of sequence tags in all the sliding windows on the particular chromosome, including sequence windows with a wide range of G/C-contents. To minimize the influence of sequencing artifacts caused by inhomogeneous G/C-content on the comparison statistics, the present invention provides a method to make a comparison of sequence tag densities within the same narrow G/C-content intervals, and combine statistic values from each G/C-content interval into one integrated value weighted by the percentage of sequence tags within each interval. This will not only allow more robust statistics by decreasing data variation caused by inhomogeneous G/C content, but also allow filtering of low quality data in low and high G/C region. It is reported that G/C poor chromosome (e.g. Chr. 4 and 13) and G/C rich chromosome (e.g. Chr. 17, 22, and 19) tend to have higher variation in sequence tag counts than chromosomes with moderate G/C levels (e.g. Chr. 9, 10 and 11), suggesting that G/C-content may also affect the quality of sequencing data. By using selected G/C-content intervals, it is possible to focus the analysis on high quality sequence data selected from a pool of mixed data. This will further increase the test sensitivity and prediction accuracy especially in low quality or low-coverage sequencing data.

### Detection of Fetal Chromosomal Abnormality in Maternal Samples Using a G/C-Adjusted Method

The present invention provides a sensitive and robust method to detect over- and under-representation of a chromosome or portion of a chromosome in a mixed sample using high throughput sequencing method. This method can minimize the influence of sequence artifacts caused by G/C-content variation, and is able to accommodate low quality and low-coverage sequencing data. This method can be applicable to determine fetal aneuploidy, fetal sex as well as adult chromosomal aneuploidy.

The present method uses shotgun high throughput sequencing technologies to detect abnormal chromosome dosage contributed by a fetal source. It may also be used to predict a male fetus by detecting the presence of Y chromosome sequences or the decrease of X chromosome dosage. The first step of the method comprises sequencing DNA in a mixed DNA sample with normally and abnormally distributed chromosomes by a high throughput sequencing method to obtain a number of sequence tags. The mixed DNA sample can be, for example, a maternal DNA sample from blood, urine or saliva, which contains both fetal and maternal DNA. The percentage of fetal DNA is usually low in maternal sample usually less than 10%. Because of the increased sensitivity of this method, it allows detection of fetal aneuploidy with lower fetal fraction. However, preparation steps to selectively enrich fetal DNA fraction can be helpful. The sequence tags needs to have sufficient length for them to be uniquely aligned to specific chromosomal locations. Depending on the high throughput sequencing platform used, the length of the sequence tags ranges from 20 to 200 bp, preferably from 20 to 100 bp. For example, Illumina sequencing machine generates 25 bp sequence tags, and Ion Torren sequencing machine generates sequence tags about 100 bp in length.

Secondly, the sequence tags are mapped to the chromosome of origin by comparing the determined sequence in the sequence tags to a reference genome. When mapping the sequence tags, one mismatch is allowed to take consideration of polymorphism between the test chromosome and the reference genome. Each chromosome is divided into non-overlapping sliding windows of predefined length and numbers of sequence tag mapped to each sliding window are counted. The length of the sliding windows can selected from 10 kb to 200 kb, preferably 20 kb to 100 kb. The sliding window is selected such that there are sufficient number of sequence tags falling into each window and there are sufficient number of sliding windows on the chromosome of interest.

Thirdly, instead of calculating the average sequence tag density for all sliding windows on a chromosome and using this average value as the chromosome dosage of this chromosome, the present invention teaches grouping all the sliding windows of a chromosome are into narrow G/C-content categories and average sequence tag density is calculated for sliding windows in each G/C-content category. For each chromosome, the number of sliding windows within different G/C-content intervals can be determined. This allows selection of one or more G/C-content intervals with majority of sliding windows for each chromosome. Alternatively, G/C-content intervals can be selected for ones with low variation. This allows selection of G/C-content intervals best fit for a particular chromosome and exclude G/C-content intervals with few sliding windows or low quality data. In a low quality sequencing situation, this makes it possible to make reliable prediction by focusing on high quality data in the entire data set. For example, chromosome 17, 22 and 19 usually have highest variation in sequence tag counts, making it difficult to make any reliable analysis. By selecting G/C-content intervals with low variation, it is possible to make reliable analysis in these problematic chromosomes by focusing on the “good” data.

The size of G/C-content interval is selected such that it is narrow enough to ensure relatively homogenous G/C-content and is wide enough to have sufficient sliding windows for robust statistic analysis. For example, the number of sliding windows in a G/C-content interval is at least 10, 20, 30, 40, 50 and more. Preferably the number of sliding windows in a G/C-content interval is at least 30 to 50. The size of G/C-content interval can be, for example, 2%, 3%, 4%, 5%, 6%, 7%, 8% or more. In a preferred embodiment, the selected G/C-content intervals are 35-40%, 40-45% and 45-50%. In another embodiment, the selected G/C-content intervals are 35-39%, 39-43%, 43-47% and 47-50%. The selected intervals are not required to be of equal size or contiguous. They can be customized to each chromosome. For each selected G/C-content interval, an average value of the sequence tag density for all the sliding windows within the G/C-content interval is determined. The average value can be an arithmetic mean, a median, or a mode of all the sequence tag densities in a G/C-content interval. This method works for complete chromosomes and large portion of a chromosome as well. The portion of the chromosome to be tested for abnormal distribution needs to be large enough to have sufficient number of sliding windows for statistic analysis. For example, the large chromosome portion needs to have at least 30 sliding windows that can be further divided into two or three G/C-content intervals.

Fourthly, the average value of each selected G/C-content interval on the chromosome of interest in the mixed DNA sample is compared to an average value of the same selected G/C-content interval of normally distributed reference chromosome(s) to obtain a statistic value. An important feature of the present invention is to compare sequence tag densities in the same G/C-content interval so as to minimize sequencing artifacts caused by G/C bias. To determine the existence of abnormal distribution of the chromosome of interest (first chromosome), the comparison can be made between different chromosomes within the same sample or between the same chromosome of different samples. In one embodiment, the average sequence tag density in each selected G/C-content interval of the first chromosome in the testing sample is compared to that of the same chromosome in a different sample where the first chromosome is known to have a normal distribution (for example, a normal maternal sample with a disomic mother and a disomic fetus). The normal maternal samples (also called negative samples) can be collected and combined together to form a normal sample pool for comparison. To account for difference in sequence tag counts in different samples, the sequence tag density needs to be normalized to the average sequence tag density for all the autosomes in each sample. The normalized average sequence density for each G/C-content interval in all the normal samples are further averaged and this averaged sequence density of each G/C-content interval for all normal sample can be used the reference value for comparison to find abnormal chromosomal distribution.

In another embodiment, the average sequence tag density in each selected G/C-content interval of the first chromosome is compared to the average sequence tag density in the same G/C-content interval of a second chromosome or chromosomes which have a normal distribution in the same test sample. The second chromosomes can be all the autosomes in the testing sample, excluding the first chromosome. This intra-sample comparison does not depend on external control sample, and eliminates the inter-sample variation, which can be a good choice for small hospitals and clinical laboratories where a large size of normal samples are not available.

The statistic methods used to test for the equality of the means of two populations are well known to a person having ordinary skills in the art of statistics and mathematic analysis. One way to do this is to use a t-test, which provides a test for the equality of the means of two normal populations with unknown variances. The t statistics to test whether the distribution of the sequence tag density of two chromosomes is the same can be calculated as the following:

$$t = \frac{\overline{{x}_{1}} - \overline{{x}_{2}}}{\sqrt{\frac{{S}_{1}^{2}}{{N}_{1}} + \frac{{S}_{2}^{2}}{{N}_{2}}}}$$

Where X̅₁ and X̅₂ is the average value of sequence tag density of all sliding windows in a G/C-content interval for first chromosome and second chromosome, respectively. S₁ and S₂ are the standard deviation of sequence tag density of all sliding windows in a G/C-content interval for first chromosome and second chromosome, respectively. N₁ and N₂ are the number of sliding windows in a G/C-content interval for first chromosome and second chromosome, respectively. To obtain a p-value from a t-table, one needs to have the t statistic and degree of freedom (t) for the t-test.

$$υ = \frac{{( {s}_{1}^{2} / {N}_{1} + {s}_{2}^{2} / {N}_{2} )}^{2}}{{( {s}_{1}^{2} / {N}_{1} )}^{2} / ( {N}_{1} - 1 ) + {( {s}_{2}^{2} / {N}_{2} )}^{2} / ( {N}_{2} - 1 )}$$

Another test that can be used is two-sample Z-test, which is used to test the equality of the means of two populations of normal distribution when the standard deviation of the population is known. When sample size is large enough (>30), the standard deviation of the sample can be used as a close approximate of the population standard deviation. The Z-score to test whether the distribution of the sequence tag density of two chromosomes is the same can be similarly calculated as the following:

$$Z = \frac{\overline{{x}_{1}} - \overline{{x}_{2}}}{\sqrt{\frac{{S}_{1}^{2}}{{N}_{1}} + \frac{{S}_{2}^{2}}{{N}_{2}}}}$$

Where X̅₁ and X̅₂ is the average value of sequence tag density of all sliding windows in a G/C-content interval for first chromosome and second chromosome, respectively. S₁ and S₂ are the standard deviation of sequence tag density of all sliding windows in a G/C-content interval for first chromosome and second chromosome, respectively. N₁ and N₂ are the number of sliding windows in a G/C-content interval for first chromosome and second chromosome, respectively. To obtain a p-value from a Z-table, one only needs a single critical value, Z-score. It is easier to operate than t-test which needs two critical values, t statistics and degree of freedom. Z-test is therefore a preferred choice when applicable. Since the number of sliding windows in a G/C-content interval is usually larger than 30, Z-test can be used for comparing the sequence tag densities of the first and second chromosome.

Once the statistic value for each G/C-content interval is obtained, they are combined by their weight into a final statistic value. The final statistic value is calculated as follows:

Final Z-score=Z₁*P₁+Z₂*P₂+Z3*P₃ . . . +Zₙ*Pₙ

Where Z₁, Z₂, . . . Zₙ is the Z-score for each G/C-content interval, and P₁, P₂, . . . Pₙ is the corresponding percentage of sliding windows in all the selected G/C-content intervals for each G/C-content interval.

Whether there is the existence of abnormal distribution of the chromosome of interest in the test sample is determined by the final weighted statistic value and predefined confidence interval. If the final weighted statistic value falls within the boundary of a predefined confidence interval (e.g. 99%), the chromosome of interest in the test sample is considered to be normal. If the final weighted statistic value falls outside the boundary of predefined confidence interval, the chromosome of interest is considered to be abnormally distributed and an aneuploid chromosome is detected in the test sample.

### Comparison of G/C-Adjusted Method and Conventional Method

69 and 46 clinical samples were sequenced using Ion Torren and Illumina sequencing platform, respectively, which were tested for fetal aneuploidy with a focus on testing for T21, T18 and T13 abnormality using both a G/C-adjusted and a non-adjusted method (conventional method). All the normal disomic samples (negative samples) were combined to be used as the normal sample for comparison. The G/C-content intervals used were 35-40%, 40-45% and 45-50%. The cutoff z-score for rejecting a null hypothesis is 3 for a confidence internal of 99.9% (one-tail).

In Illumina samples, there were 30 of normal samples, 1 of T13 sample, 8 of T18 and 7 of T21 samples. As shown in table 1 and summarized in table 4, the G/C-adjusted method of the invention was able to detect 30 out of 30 normal samples, 8 out of 8 T18 samples and 7 out of T21 samples. It barely missed the one T13 sample, but the T13 sample has a Z-score of 2.9 on chromosome 13 with a confidence interval of 99.8% to reject the null hypothesis. This T13 abnormality can also be detected by the G/C-adjusted method if the confidence interval is set a bit lower. However, the conventional method missed detecting the T13 abnormality plus two T18 abnormalities.

The difference between these two methods in detection sensitivity and accuracy is much more significant in Ion Torren samples, where there were less sequence reads and the sequence data quality tended to be lower. In Ion Torren samples (Table 2 and 3), the G/C adjusted method of the invention was able to detect 59 out of 59 normal samples, 4 out of 4 T13 samples, 2 out of 2 T18 samples, and 4 out of 4 T21 samples. On the other hand, the conventional method missed detecting 4 out of 4 T13 samples and 3 out of 4 T21 samples. In addition, the conventional method made a false negative call. It is very clear from these data that the G/C-adjusted method of the invention performs much better for lower quality data than the conventional method does.

### Computer Program for Detecting Fetal Chromosomal Abnormality

In one embodiment, the present invention provides a computer program that implements the method described above to detect an abnormal distribution of chromosome or chromosome portion of interest in a mixed DNA sample of normally and abnormally distributed chromosomes by counting sequence tags obtained from a high throughput sequencing of the mixed DNA sample. The computer program takes sequence tag reads of a high throughput sequencing of the mixed DNA sample as input data. The sequence reads can be from any high throughput sequencing platform. The most common ones are Illumia and Ion Torren sequencing platform.

The program divides each chromosome of a reference genome into non-overlapping windows and maps the input sequence tags to these windows. Customers can choose if they want exact mapping or mapping with mismatches.

The program then counts numbers of sequence tags falling in every window on each chromosome. The number of sequence tags in each window is normalized to the average value of sequence tag counts in all the windows of all the autosomes in the sample. The computer can group the windows based on selected G/C-content intervals for each chromosome and calculate the mean or median value of sequence tag counts in each selected G/C-content interval. The computer program makes comparison of the mean or median sequence tag counts between any two chromosomes within the same sample or compare sequence tag counts of chromosome of interest between the test sample and normal samples stored in the computer system. The computer can also make comparison between one chromosome vs. multiple chromosomes, or between one sample vs. multiple normal samples. The computer program outputs a final weighted statistic value based on individual statistic value of each selected G/C-content interval and make a decision call on the existence of chromosome abnormality based on a pre-selected confidence interval. The computer program can be used to analyze sequencing data from difference sequencing platforms, for example, Illumina high throughput sequencing platform and Ion Proton high throughput sequencing system from Thermal Fishier Scientific. It supports separate user account and data sharing among different users.

### EXAMPLES

The following examples are provided for illustration purposes, are not intended to limit the scope of the invention, which is limited only by the claims.

### Example 1

### Relationship Between Parameters in High Throughput Sequencing

In order to search for directions to improve the sensitivity and accuracy of methods for analyzing high throughput sequencing data, we studied correlations between different parameters of the sequencing. The selected parameters are G/C content of sequences (GC), Degree of freedom (df), mean of sequence tag reads (mean), median of sequence tag reads (median), weighted mean of sequence tag reads (weight_mean) and weighted median of sequence tag reads (weight_median).

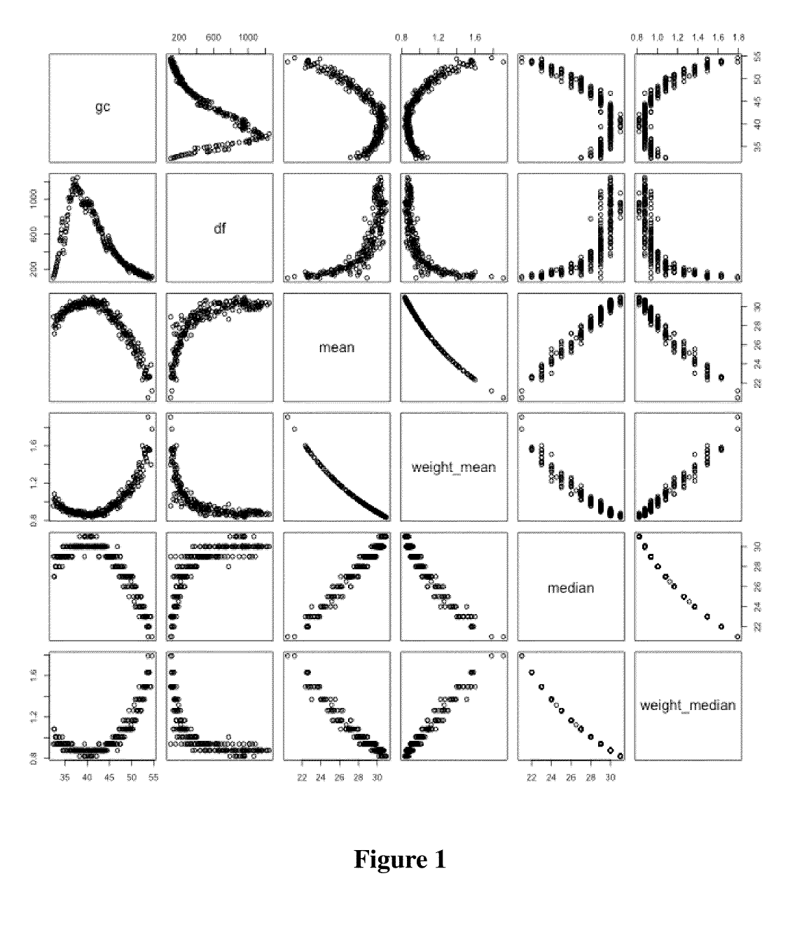

FIG. 1 is a scatter plot showing the relationship between any two parameters chosen from GC, df, mean, median, weight_mean and weight_median. The scatter plot shows that mean, median, weight_mean, and weight_median linearly correlate well with each other, suggesting using any of this parameter in an analysis may not make a big difference. But GC has a non-linear relationship with all the other parameters. The influence of G/C content in sequence read analysis cannot be easily compensated by any linear method. Consistent with other's finding, we conclude that correcting the influence caused by sequence G/C-content on sequence tag counts and final analysis output is important for improving high throughput data analysis.

### Example 2

### Distribution of Human Genomic Sequence Counts within Different Intervals of G/C-Contents

The whole human genome (hg19) was divided into large fragments/bins of 20 kb, which were grouped into five different categories based on their sequence G/C-contents. The G/C-contents for the five categories were 30-35%, 35-40%, 40-45%, 45-50% and 50-55%. The x-axis is the count of sequence tags in a bin (a measuring window) and the y-axis is the number of bins having the corresponding sequence tag counts. The figure shows that the majority of sequences fall in the three G/C categories: 35-40%, 40-45% and 45-50%, and different groups of sequences have different peak values. The peak value of the high G/C group (50-55%) deviates most from the rest of the groups. This is also consistent with findings from other researchers that chromosomes with high G/C content tend to have much more variation in sequence reads than chromosomes with moderate G/C contents. High G/C content may cause sequencing errors and inconsistency by interfering with denaturing and annealing process during PCR amplification. The low (30-35%) or high (50-55%) G/C groups tend to have sequencing data with lower quality and higher variation. In majority of human chromosomes, the total percentage of these two categories of sequence bins is relatively small. It is advantageous to leave out sequence data from low and high G/C groups and focus the analysis on the high quality data from sequence groups with moderate G/C content. This results in more sensitive analysis and allows accurate detection of abnormal distribution from a lower quality data set.

### Example 3

### Detection of Fetal Chromosome Abnormality in Maternal Samples

Blood samples were drawn from pregnant women participated in the study for detecting fetal chromosome abnormality. Informed consent was obtained from each participant prior to blood draw. Karyotype analysis was performed via aminicentesis or chrionic villus sampling to confirm fetal karyotype. 30 normal, 1 T13, 8 T18 and 7 T21 singleton pregnancies were included for study using an Illumina sequencing platform. 49 normal, 4 T13, 2 T18 and 4 T21 singleton pregnancies were included for study using an Ion Torren sequencing platform. Blood samples were centrifuged to remove cellular components and obtain cell-free plasma. Cell-free circulating DNA was extracted from plasma samples using QIAmp Circulating Nucleic Acid Kit (Qiagen, Valencia, Calif.) according to manufacturer's instruction. A total of 69 cell-free plasma DNA samples were used for library preparation and sequenced on the Illumina sequencing platform. A total of 46 cell-free plasma DNA samples were used for library preparation and sequenced on the Ion Torren sequencing platform.

The sequencing reads were analyzed and tested for fetal aneuploidy using both a G/C-adjusted and a conventional method. The fetal aneuploidy detection was focused on three common trisomies in human, T21, T18 and T13. All the normal disomic samples (negative samples) were combined to be used as the normal sample for comparison. The G/C-content intervals used were 35-40%, 40-45% and 45-50%. The statistic method used for comparison the difference in the median sequence tag density of two chromosome is z-test. The cutoff z-score for rejecting a null hypothesis is 3 for a confidence internal of 99.9% (one-tail).

In Illumina samples, there were 30 of normal samples, 1 of T13 sample, 8 of T18 and 7 of T21 samples. As shown in table 1 and summarized in table 4, the G/C-adjusted method of the invention was able to detect 30 out of 30 normal samples, 8 out of 8 T18 samples and 7 out of T21 samples. It barely missed the one T13 sample, but the T13 sample has a Z-score of 2.9 on chromosome 13 with a confidence interval of 99.8% to reject the null hypothesis. This T13 abnormality can also be detected by the G/C-adjusted method if the confidence interval is set a bit lower. On the other hand, the conventional method detects 30 out of 30 normal samples, 6 out of 8 T18 samples and 7 out of T21 samples. The conventional method missed detecting the T13 abnormality plus two T18 abnormalities.

The difference between these two methods in detection sensitivity and accuracy is much more significant in Ion Torren samples, where there were less sequence reads and the sequence data quality tended to be lower. In Ion Torren samples (Table 2 and 3), the G/C adjusted method of the invention was able to detect 59 out of 59 normal samples, 4 out of 4 T13 samples, 2 out of 2 T18 samples, and 4 out 4 T21 samples. The G/C-adjusted method has a 100% prediction accuracy. On the other hand, the conventional method was able to detect only 1 out of 4 T21 sample and 2 out of 2 T18 samples. It missed detecting 4 out of 4 T13 samples and 3 out of 4 T21 samples. In addition, the conventional method made a false negative call. It is very clear from these data that the G/C-adjusted method of the invention performs better than the conventional method especially in analysis of lower quality data.

### Example 4

### Computer Program iNIPT (Non-Invasive Prenatal Test) for Detecting T21, T18 and T13 Fetal Abnormality

An online computer program iNIPT was developed to implement the method of the invention to detect an abnormal distribution of chromosome or chromosome portion of interest in a maternal DNA sample. The program was designed for detecting T21, T18 and T13 fetal trisomies, but can also adapted to detect other abnormal fetal chromosomes. This program allows selection of G/C-adjusted or conventional algorithm for analyzing the sequence tags obtained from any sequencing platform.

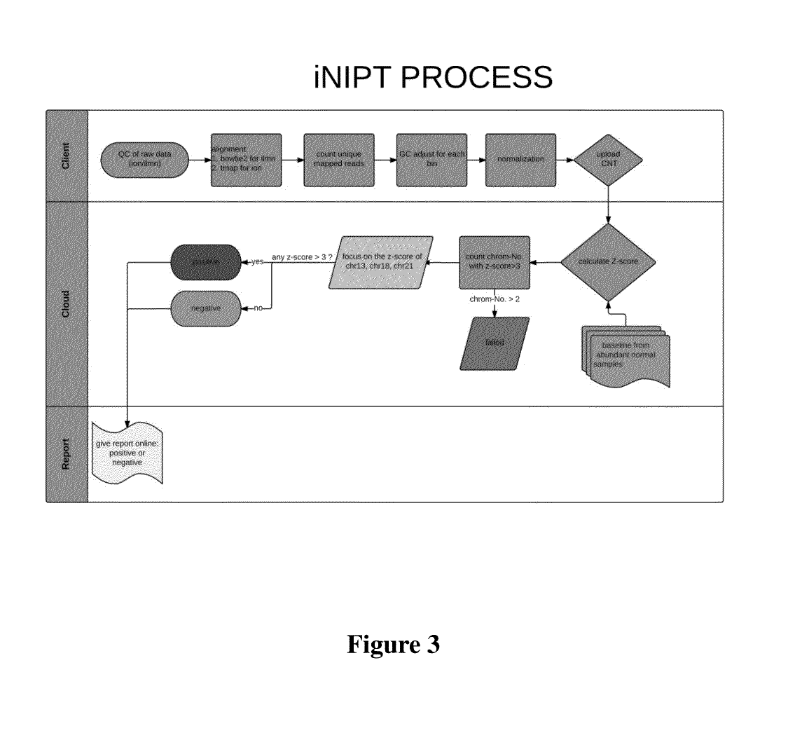

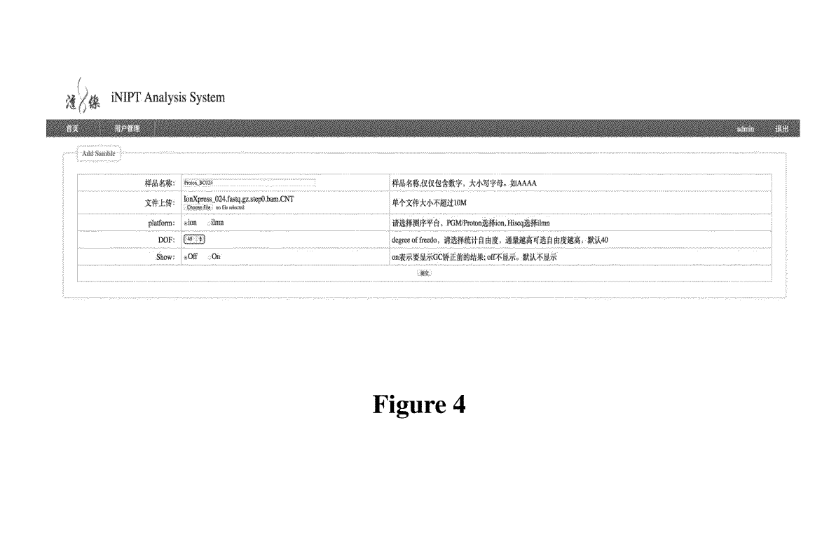

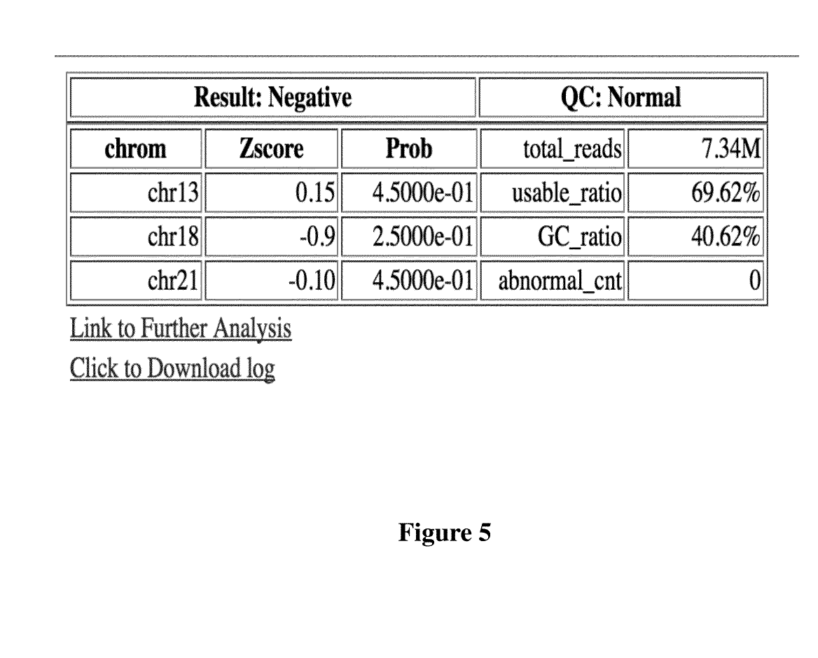

The computer program takes sequence tag reads of a high throughput sequencing of the mixed DNA sample as input data. The sequence reads can be from any high throughput sequencing platform. The most common ones are Illumia and Ion Torren sequencing platform. The program outputs a final statistic value and make a decision call on the existence of chromosome abnormality based on a pre-selected confidence interval. FIG. 3 shows a diagram of computer program (iNIPT) workflow for detecting fetal Trisomy 13, 18 and 21 in a maternal sample. FIG. 4 shows the launching page with parameters setting of computer program (iNIPT). FIG. 5 shows the output and report page of iNIPT. The output page shows the G/C-adjusted Z-score for chromosome 21, 18 and 13 and corresponding probabilities that accept the null hypothesis (the chromosome is normal).

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A method of detecting an abnormally distributed chromosome of interest in a DNA sample containing normally and abnormally distributed chromosomal DNAs, comprising the steps of:
a, Sequencing DNA in said DNA sample by a high throughput sequencing method to obtain a number of sequence tags of sufficient length to be assigned to a chromosome location of a genome;
b, Mapping the sequence tags to the chromosome of origin, wherein each chromosome is divided into non-overlapping sliding windows of predefined length;
c, Determining sequence tag density mapped to each sliding window;
d, Determining G/C-content of each sliding window on the chromosome of interest;
e, Selecting one or more G/C-content intervals of the sliding windows on the chromosome of interest;
f, Determining a mean or median value of said sequence tag density for all the sliding windows in each selected G/C-content interval;
g, Comparing said mean or median value of each selected G/C-content interval on the chromosome of interest in said DNA sample to a mean or median value of the same selected G/C interval of normally distributed reference chromosome(s) to obtain a statistic value, which is combined to obtain a weighted statistic value; and
h, Determining the existence of an abnormal distribution of the chromosome of interest in said DNA sample based on said weighted statistic value.

2. The method of claim 1, wherein said DNA sample is cell-free circulating DNA in maternal blood having fetal and maternal DNA.

3. The method of claim 1, wherein the abnormally distributed chromosome is an aneuploid chromosome.

4. The method of claim 1, wherein the sequence tags are of a length from 20 to 200 bp.

5. The method of claim 1, wherein the sliding window is of a length from 10 kb to 100 kb.

6. The method of claim 1, wherein the selected G/C-content interval is 35% to 50%.

7. The method of claim 1, wherein the selected G/C-content intervals are 35%-40%, 40-45%, 45-50%.

8. The method of claim 1, wherein the statistic value is a z-score.

9. The method of claim 1, wherein an abnormal distribution of said chromosome of interest is detected when said weighted statistic value indicates that the distribution of said chromosome of interest is out of predefined confidence interval.

10. The method of claim 1, wherein the sequence tag densities are normalized to an average sequence tag density for all the autosomes in said DNA sample.

11. The method of claim 1, wherein said normally distributed reference chromosome is the same chromosome of interest in a sample having normal distribution of the chromosome of interest.

12. The method of claim 1, wherein said normally distributed reference chromosome is a chromosome having normal distribution in the same sample, which is different from the chromosome of interest.

13. The method of claim 1, wherein said normally distributed reference chromosome is more than one chromosomes having normal distribution in the same sample excluding the chromosome of interest.
